(19) United States
(12) Patent Application Publication
(10) Pub. No.: US 2024/0428914 A1
(43) Pub. Date: Dec. 26, 2024

(54) SYSTEMS AND METHODS FOR PROVIDING MEDICATION TREATMENTS BASED ON PHARMACOGENETIC TESTING

(71) Applicant: VDS723, LLC, Scottsdale, AZ (US)

(72) Inventor: Divyen Patel, Schoharie, NY (US)

(21) Appl. No.: 18/635,944

(22) Filed: Apr. 15, 2024

Related U.S. Application Data
(60) Provisional application No. 63/460,018, filed on Apr. 17, 2023.

(51) Int. Cl. G16H 20/10; G16B 20/00; G16H 15/00

(52) U.S. Cl. CPC G16H 20/10; G16B 20/00; G16H 15/00

(57) ABSTRACT

Systems and methods for providing medication treatments based on pharmacogenetic testing may include one or more processors that store a database comprising a plurality of slots. The slots respectively may corresponding to data indicative of a gene marker and a treatment efficacy. The data indicative of the gene markers and treatment efficacies may be derived from a plurality of data sources. The processor(s) may receive an input extracted from a sample obtained from a patient, the input including a set of genetic sequences of the patient. The processor(s) may correlate at least one of the set of genetic sequences from the input to a gene marker of a slot from the database, to determine a treatment efficacy of a treatment option for the patient. The processor(s) may generate a report indicating the treatment efficacy of the treatment option for the patient.

### CROSS-REFERENCE TO RELATED APPLICATIONS

This application claims the benefit of and priority to U.S. Provisional Application No. 63/460,018, filed Apr. 17, 2023, the contents of which are incorporated by reference in its entirety.

### BACKGROUND

Pharmacogenetic (also referred to as pharmacogenomic) testing is a type of genetic testing that analyzes an individual's genetic makeup to predict how the individual may respond to certain medications. Pharmacogenetic testing looks for specific genetic variations that can affect how a person's body metabolizes drugs or medications (i.e., how effectively the medications are absorbed and eliminated by the person's body). The results of pharmacogenetic testing can help doctors make more informed decisions about which medications to prescribe and at what dosages. For example, some genetic variations may make a person more or less likely to experience side effects from a particular medication or may affect efficacy of the medication for the person.

While pharmacogenetic testing is widely available, the ability to determine whether medications to prescribe and at what dosages is challenging because each human DNA molecule contains many genes, and the human genome is estimated to contain approximately 20,000-25,000 genes. Moreover, there are 3 billion base pairs of DNA in the human genome that are organized into 23 distinct, physically separate microscopic units called chromosomes. Such information along with associated responsiveness of people with specific genes to medications is currently available in several public databases, such as PharmGKB, Pharm Var, etc., but due to the databases containing vasts amounts of genomic data and associated medication information (millions of data points), the ability to readily and reliably access the information is extremely challenging. To complicate the situation, several of the public databases have different data formats and use different computer languages from the others. In addition, the public databases are distributed in different research facilities, universities, and so on, thereby making the ability to utilize the data commercially infeasible. Hence, being able to correlate the genetic makeup of a person and prescribe proper or optimized medications for that person so as to provide effective personalized medicine has been commercially challenging. As such, there is a need to provide a feasible way to correlate an individual's genetic makeup with billions of data points to determine and prescribe medication treatments, so as to enable physicians to effectively and confidently provide personalized medicine.

### SUMMARY

To provide physicians with feasible way to use pharmacogenetic testing in providing customized medication regimens when prescribing medications to individual patients, systems and processes that utilizes a data process for filtering or curate (e.g., multiple filtering processes) millions of data points from public databases may be used. In providing personalized medicine with predictable outcomes, results of a pharmacogenetic test may be applied to the multi-filtered data points to determine whether medications are able to be metabolized by the individual patients, thereby enabling physicians to provide predictable medication regimens (e.g., specifying medication and dosages). The data filtering process may be a big data process that may be applied to publicly available worldwide databases to filter data points to a more manageable number (e.g., hundreds of thousands, such as about 830,000) of data points and in a format that is readily applicable to an output of a diversity array. A feasible and economical system to design personalized treatments with medicines with predictive patient outcomes may therefore be provided utilizing the principles described herein. In addition to the system being used to predict responsiveness to a medication, the use of the system and processes may further provide for a patient's disease risk.

In one aspect, this disclosure is directed to a computer-implemented method. The method may include storing, by one or more processors in a non-transitory memory, a database comprising a plurality of slots, the slots respectively corresponding to data indicative of a gene marker and a treatment efficacy, the data indicative of the gene markers and treatment efficacies derived from a plurality of data sources. The method may include receiving, by the one or more processors, an input extracted from a sample obtained from a patient, the input including a set of genetic sequences of the patient. The method may include correlating, by the one or more processors, at least one of the set of genetic sequences from the input to a gene marker of a slot from the database, to determine a treatment efficacy of a treatment option for the patient. The method may include generating, by the one or more processors, a report indicating the treatment efficacy of the treatment option for the patient.

In some embodiments, the report identifies, for a plurality of conditions, a plurality of respective medication treatments, and for each respective medication treatment, a phenotype guideline indicating a predicted metabolizer and an evidence guideline indicating a degree of evidence from the database originating from a data source of the plurality of data sources. In some embodiments, the method includes determining, by the one or more processors, for a medication treatment of the plurality of respective medication treatments, that the phenotype guideline satisfies a threshold predicted metabolizer. The method may include generating, by the one or more processors, the report to prompt selection of an alternative medication treatment other than the medication treatment based on the phenotype guideline satisfying the threshold predicted metabolizer.

In some embodiments, the method includes receiving, by the one or more processors, from the treating professional, an indication of a plurality of conditions of the patient and, for each of the plurality of conditions, a corresponding medication treatment identified by the treating professional for treating the respective condition. In some embodiments, the report identifies, for the corresponding medication treatment for the plurality of conditions, a risk profile associated with the corresponding medication treatment. In some embodiments, the risk profile for each medication treatment identifies a phenotype guideline indicating a predicted metabolizer and an evidence guideline indicating a degree of evidence from the database. In some embodiments, the method includes determining, by the one or more processors, the risk profile for each medication treatment by performing a look-up in the database using an identifier associated with a gene sequence of the set of gene sequences of the patient, to identify a gene marker matching the identifier associated with the gene sequence, the gene marker indicating an efficacy of the corresponding medication treatment for patients having the gene sequence.

In some embodiments, the method includes populating, by the one or more processors, the database using the data compiled from the plurality of data sources. In some embodiments, populating the database includes extracting, by the one or more processors, datasets from the plurality of data sources, each dataset including a plurality of data points, each dataset from the plurality of data sources including at least some data of the plurality of slots. Populating the database may include compiling, by the one or more processors, the datasets from the plurality of data sources into a plurality of first slots, the data points of a respective dataset being assigned to a corresponding first slot of the plurality of first slots. Populating the database may include combining at least some slots into a grouped slot, based on a correspondence between a data point of one slot and another data point of another slot. Populating the database may include generating a plurality of second slots to include the plurality of first slots including the combined group slot. The database may be populated with the plurality of second slots.

In another aspect, this disclosure is directed to a system that may include a non-transitory memory storing a database comprising a plurality of slots, the slots respectively corresponding to data indicative of a gene marker and a treatment efficacy, the data indicative of the gene markers and treatment efficacies derived from a plurality of data sources. The system may include one or more processors configured by machine-readable instructions to receive an input extracted from a sample obtained from a patient, the input including a set of genetic sequences of the patient. The one or more processors may be configured by machine-readable instructions to correlate at least one of the set of genetic sequences from the input to a gene marker of a slot from the database, to determine a treatment efficacy of a treatment option for the patient. The one or more processors may be configured by machine-readable instructions to generate a report indicating the treatment efficacy of the treatment option for the patient.

In some embodiments, the report identifies, for a plurality of conditions, a plurality of respective medication treatments, and for each respective medication treatment, a phenotype guideline indicating a predicted metabolizer and an evidence guideline indicating a degree of evidence from the database originating from a data source of the plurality of data sources. In some embodiments, the one or more processors are further configured by machine-readable instructions to determine, for a medication treatment of the plurality of respective medication treatments, that the phenotype guideline satisfies a threshold predicted metabolizer. The one or more processors may be configured by machine-readable instructions to generate the report to prompt selection of an alternative medication treatment other than the medication treatment based on the phenotype guideline satisfying the threshold predicted metabolizer.

The processor(s) may further be configured by machine-readable instructions to receive, from the treating professional, an indication of a plurality of conditions of the patient and, for each of the conditions, a corresponding medication treatment identified by the treating professional for treating the respective condition. In some embodiments, the report identifies, for the corresponding medication treatment for the plurality of conditions, a risk profile associated with the corresponding medication treatment. In some embodiments, the risk profile for each medication treatment identifies a phenotype guideline indicating a predicted metabolizer and an evidence guideline indicating a degree of evidence from the database.

The processor(s) may further configured by machine-readable instructions to populate the database using the data compiled from the plurality of data sources. In some embodiments, populating the database includes extracting, by the one or more processors, datasets from the plurality of data sources, each dataset including a plurality of data points, each dataset from the plurality of data sources including at least some data of the plurality of slots. Populating the database may include compiling, by the one or more processors, the datasets from the plurality of data sources into a plurality of first slots, the data points of a respective dataset being assigned to a corresponding first slot of the plurality of first slots. Populating the database may include combining at least some slots into a grouped slot, based on a correspondence between a data point of one slot and another data point of another slot. Populating the database may include generating a plurality of second slots to include the plurality of first slots including the combined group slot. The database may be populated with the plurality of second slots.

In yet another aspect, this disclosure is directed to a non-transitory computer readable medium storing instructions that, when executed by one or more processors, cause the one or more processors to access a database comprising a plurality of slots, the slots respectively corresponding to data indicative of a gene marker and a treatment efficacy, the data indicative of the gene markers and treatment efficacies derived from a plurality of data sources. The instructions may cause the one or more processors to receive an input extracted from a sample obtained from a patient, the input including a set of genetic sequences of the patient. The instructions may cause the one or more processors to correlate at least one of the set of genetic sequences from the input to a gene marker of a slot from the database, to determine a treatment efficacy of a treatment option for the patient. The instructions may cause the one or more processors to The instructions may cause the one or more processors to generate a report indicating the treatment efficacy of the treatment option for the patient.

In some embodiments, the instructions cause the one or more processors to populate the database using the data compiled from the plurality of data sources.

### BRIEF DESCRIPTION OF THE DRAWINGS

Illustrative embodiments of the present invention are described in detail below with reference to the attached drawing figures, which are incorporated by reference herein and wherein:

FIG. 1 is a diagram of a patient and a treating professional, according to a context of the present disclosure.

FIG. 2 is a block diagram of a system for pharmacogenetic testing, according to an example implementation of the present disclosure.

FIG. 3 is a block diagram of a database compiled or derived from a plurality of data sources, according to an example implementation of the present disclosure.

FIG. 4 is a diagram of a sample obtained from the patient of FIG. 1 and provided to a diversity array of the system of FIG. 3, according to an example implementation of the present disclosure.

FIG. 5 is a diagram of tables illustrating gene sequences, medication treatment names, and medication treatment classifications, according to example implementations of the present disclosure.

FIG. 6 is a diagram of an example patient report generated by the system of FIG. 2, according to an example implementation of the present disclosure.

FIG. 7 is a diagram of a table that may be appended to the patient report of FIG. 6, indicating treatment efficacies of various medication treatments, according to an example implementation of the present disclosure.

FIG. 8 depicts an example block diagram of an example computer system.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The practice of the present technology will employ, unless otherwise indicated, conventional techniques of organic chemistry, pharmacology, immunology, molecular biology, microbiology, cell biology and recombinant DNA, which are within the skill of the art. Sec, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs.

“An effective amount” or “therapeutically effect amount” intends to indicate the amount of a compound or agent administered or delivered to the patient which is most likely to result in the desired response to treatment. The amount is empirically determined by the patient's clinical parameters including, but not limited to the Stage of disease, age, gender, histology, and likelihood for tumor recurrence.

A “patient” as used herein intends an animal patient, a mammal patient or yet further a human patient. For the purpose of illustration only, a mammal includes but is not limited to a simian, a murine, a bovine, an equine, a porcine or an ovine subject.

The term “clinical outcome”, “clinical parameter”, “clinical response”, or “clinical endpoint” refers to any clinical observation or measurement relating to a patient's reaction to a therapy. Non-limiting examples of clinical outcomes include.

The term “identify” or “identifying” is to associate or affiliate a patient closely to a group or population of patients who likely experience the same or a similar clinical response to a therapy.

The term “selecting” a patient for a therapy refers to making an indication that the selected patient is suitable for the therapy. Such an indication can be made in writing by, for instance, a handwritten prescription or a computerized report making the corresponding prescription or recommendation.

“Detecting” as used herein refers to determining the presence of a nucleic acid of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

“TaqMan® PCR detection system” as used herein refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, 5′-exonuclease activity of the Taq polymerase cleaves the probe thereby allowing the donor fluorophore to emit fluorescence which can be detected.

As used herein, the term “sample” or “test sample” refers to any liquid or solid material containing nucleic acids such as miRNA, e.g., peripheral blood mononuclear cells (PBMCs). In suitable embodiments, a test sample is obtained from a biological source (i.e., a “biological sample”), such as cells in culture or a tissue sample from an animal, preferably, a human. In an exemplary embodiment, the sample is a biopsy sample.

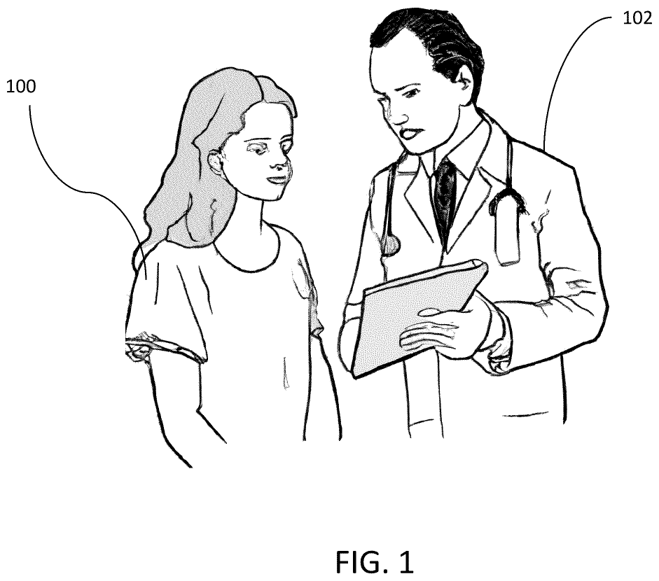

Referring to FIG. 1, depicted is a diagram of a patient 100 and a treating professional 102, according to a context of the present disclosure. Some treating professionals 102 may request, prescribe, or otherwise cause a patient 100 to have a pharmacogenetic (PGx) test for various purposes. For example, the patient 100 may have a pharmacogenetic test to help the treating professional 102 make more informed decisions about which medications to prescribe and at what dosages. By analyzing the genetic makeup of the patient 100, a PGx test can identify specific genetic variations that can affect how the patient's body metabolizes medication treatments or drugs, how effectively such medication treatments are absorbed, and how medication treatments are eliminated from the body. Some patients 100 may have experienced adverse reactions to medications in the past and want to avoid similar problems in the future. Alternatively, some patients 100 may have a family history of medication-related problems or genetic conditions that affect drug metabolism. PGx testing can also be used to optimize treatment for chronic conditions such as depression, anxiety, or pain management, where finding the right medication and dosage can be challenging.

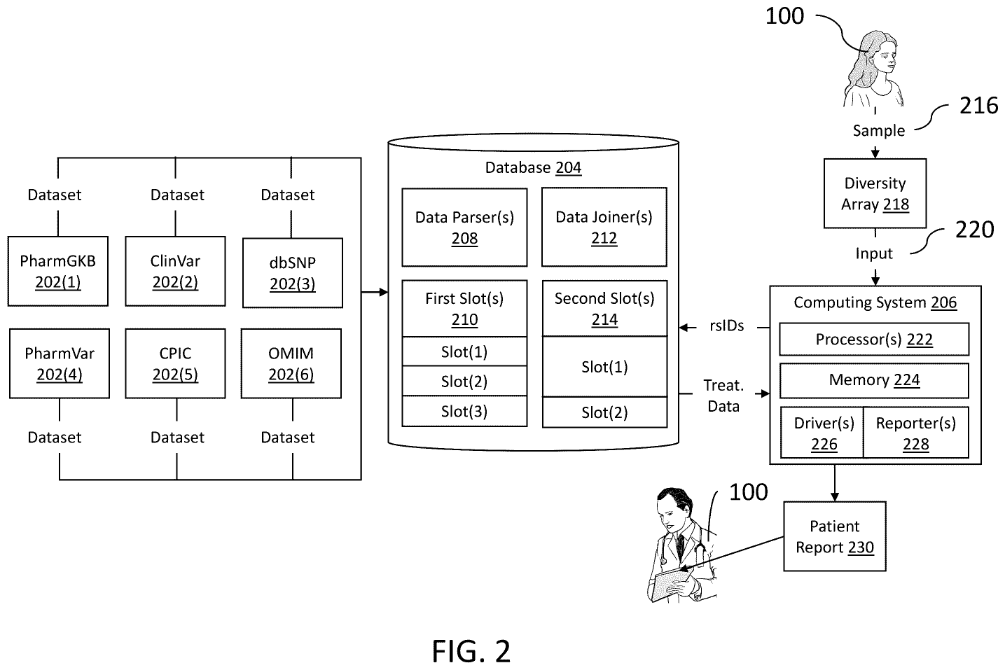

Referring to FIG. 2, a block diagram of a system 200 for pharmacogenetic (PGx) testing, according to an example implementation of the present disclosure. As shown in FIG. 2, the system 200 may include a plurality of data sources 202, a database 204, and a computing system 206. One challenge with PGx testing is diverse data across a plurality of data sources. For example, such data sources may include PharmGKB 202 (1), ClinVar 202 (2), dbSNP 202 (3), Pharm Var 202 (4), CPIC 202 (5), and OMIM 202 (6), to name a few data sources. Several of the data sources 202 may provide datasets in different formats, may include duplicative data, utilize different programming languages, be located in distributed locations, and the like. Thus, providing a broadbase PGx test can be challenging when attempting to harmonize data across the datasets from the different data sources 202.

Pharmacogenomics Knowledgebase (or PharmGKB 202 (1)) is a publicly available resource that provides information on how genetic variations can impact drug response. PharmGKB 202 (1) is a comprehensive database that collects, maintains, and disseminates information related to pharmacogenomic research, including drug-gene-disease relationships, drug labels with pharmacogenomic information, and clinical guidelines. PharmGKB 202 (1) also offers a knowledge-based clinical annotation tool that can help identify relevant genetic variations and their impact on drug response. PharmGKB 202 (1) may be regularly updated with new information and research findings. PharmGKB 202 (1) can output data in various file types, such as TSV and/or JSON. For example, PharmGKB 202 (1) can output variant and clinical file annotations (clinicalAnnotations), clinical guideline annotations (guideAnnotations), and variant, gene, and drug relationships (relationships).

Similarly, ClinVar 202 (2) is a publicly available database that collects and archives information on genetic variations and their clinical significance. ClinVar 202 (2) is a centralized resource that collects data from a variety of sources, including clinical laboratories, research studies, and individual submissions. Clin Var 202 (2) provides information on genetic variants and their association with disease, drug response, and other clinical outcomes. Clin Var 202 (2) collects information on the interpretation of genetic variants, including variant classifications and the evidence supporting those classifications. ClinVar 202 (2) also includes information on the prevalence of genetic variants in different populations and their clinical significance. ClinVar 202 (2) can output data in a VCF format. For example, ClinVar 202 (2) may output ClinVar GRCh38.

Database of Single Nucleotide Polymorphisms (dbSNP) 202 (3) is a publicly available database that catalogs and archives information on genetic variations, including single nucleotide polymorphisms (SNPs) and small insertions/deletions (indels), dbSNP 202 (3) is a comprehensive resource that collects data from a variety of sources, including research studies, genetic databases, and individual submissions, dbSNP 202 (3) provides information on genetic variations and their prevalence in different populations, as well as functional annotations and clinical significance. One of the features of dbSNP is the ability to assign unique identifiers (rsIDs) to genetic variations, which allows for standardized and consistent reporting of genetic variation across different studies and databases.

Pharmacogene Variation (Pharm Var) 202 (4) is a freely accessible database of pharmacogenomic variation that collects information on genetic variations that have been shown to impact drug response, such as variations in drug-metabolizing enzymes, transporters, and drug targets. Pharm Var 202 (4) includes information on variant nomenclature, allele frequencies, and functional annotations, as well as drug-gene annotations and recommended clinical guidelines. Pharm Var 202 (4) also includes a comprehensive set of standard terms and definitions to ensure consistent reporting and interpretation of pharmacogenomic data. Pharm Var 202 (4) is designed to be complementary to other pharmacogenomic resources such as Clin Var 202 (2) and dbSNP 202 (3). Pharm Var 202 (4) may output data in VCF format (similar to ClinVar 202 (2)), including allele definitions.

Clinical Pharmacogenetics Implementation Consortium (CPIC) 202 (5) provides guidelines for the use of pharmacogenetic testing in clinical practice. The CPIC guidelines from CPIC 202 (5) are evidence-based and developed through a process that involves a systematic review of the available literature and expert consensus. CPIC 202 (5) provides recommendations on how to interpret pharmacogenetic test results and how to use that information to optimize medication selection, dosing, and monitoring for individual patients. CPIC guidelines cover a wide range of drug-gene pairs and are regularly updated to reflect the latest research findings. CPIC 202 (5) may output data as an SQL format.

Online Mendelian Inheritance in Man (OMIM) 202 (6) is a publicly available database that catalogs and archives information on human genes and genetic disorders. OMIM 202 (6) provides comprehensive information on genetic disorders, including their clinical features, inheritance patterns, and molecular genetics. OMIM 202 (6) also includes information on the genes and genetic variations associated with thes disorders, as well as their functions and clinical significance. OMIM 202 (6) may output data in a TXT format. The data may include a gene map, MIM titles, and a morbid map.

Below is a table illustrating the different data sources, files, and file types.

TABLE 1
Data Sources and File Information

| Data Source | Files (Name) | File Type |
|---|---|---|
| PharmGKB | Variant and Clinical Annotations | TSV, |
| | (clinicalAnnotations) | JSON |
| | Clinical Guideline Annotations | |
| | (guidelineAnnotations) | |
| | Variant, Gene, and Drug Relationship | |
| | (relationships) | |
| Clin Var | ClinVar GRCh38 (clinvar_[yyyymmdd]) | VCF |
| CPIC | CPIC Database Dump (cpic_db_dump-[version]) | SQL |
| Pharm Var | Allele Definitions (pharmvar-[version]) | VCF |
| OMIM | Gene Map, MIM Titles, Morbid Map | TXT |
| | (mimTitles, genemap2, morbidmap) | |

Each of these databases or data sources 202 may provide valuable information in assisting a treating professional 102 with performing a pharmacogenetic (PGx) analysis and test on a patient 100. However, each of the data sources 202 may provide data in different respective formats, and therefore accessing each of the data sources 202 may be logistically challenging. Further, each of the data sources 202 may have vast amounts of data (e.g., on the order of >1B data points), which in combination may not be feasible or practical to parse for providing personalized medicine. For example, a patient's PGx test may involve cross-referencing 50,000 SNPs from a patient sample against data in each of the plurality of data sources 202. To execute separate queries for each data source 202 would require multiple iterations of queries in different formats, which can cause delay in delivery of results, greater storage requirements to maintain separate code for executing the separate queries, and could result in redundant results. According to the systems and methods of the present solution, the system 200 may be configured to compile and curate the database 204 to expedite delivery of results, while reducing the number of queries for producing results.

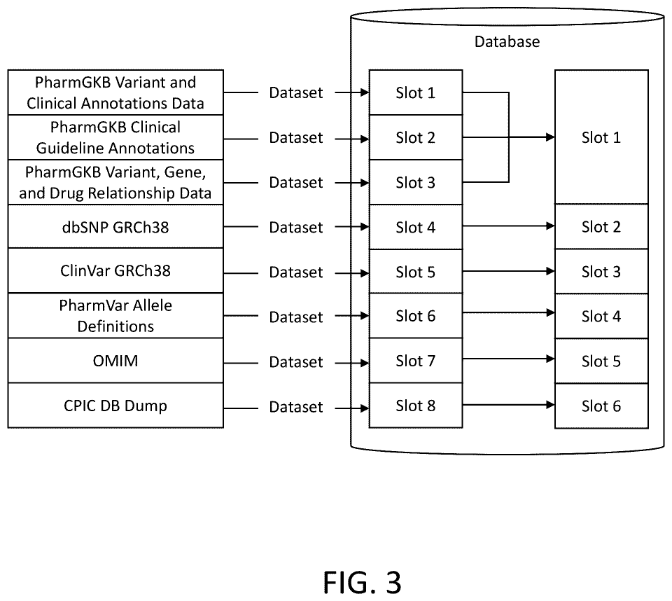

Referring to FIG. 2 and FIG. 3, in some embodiments, the system 200 may include one or more data parser(s) 208. The data parser(s) 208 may be or include any device, component, element, or hardware designed or configured to collect, extract, retain, receive, or otherwise compile the datasets from the data sources 202. In some embodiments, the system 200 may include multiple data parsers 208. For example, the system 200 may include a data parser for each file type (e.g., one data parser 208 configured to query data sources 202 that output data in SQL, another data parser 208 configured to query data sources 202 that output data in TSV or JSON, another data parser 208 configured to query data sources 202 that output data in VCF, etc.). As another example, the system 200 may include a data parser 208 for each data source 202 (e.g., a parser for PharmGKB [parse_PGKB.R], a parser for CPIC, a parser for OMIM, a parser for dbSNP), etc.). An example of the data parser 208 for PharmGKB is below, though it should be understood that similar data parsers 208 may be deployed and executable for each of the plurality of data sources 202.

### Executable Code for Parser for PharmGKP

```
parse_input <− function(data_path) {
  data <− read_lines(data_path)
  find_comments <− map_lgl(data, ~ str_detect(.x, ″#″))
  position <−
    which(find_comments == TRUE)[length(which(find_comments ==
    TRUE))]
  data <− read_tsv(data_path, skip = position) |>
    dplyr::filter(!str_detect(id, ″AFFX″) | str_detect(id, ″BGP″)) |>
    dplyr::rename(‘Probe Set ID‘ = id)
}
parse_annotation <− function(data_path) {
  pmda_annotation <− read_csv(data_path,
        skip = 20) |>
    dplyr::filter(‘dbSNP RS ID‘ != ″---″) |>
    dplyr::select(‘Probe Set ID‘,
      ‘dbSNP RS ID‘,
      Chromosome,
      ‘Physical Position‘,
      ‘Position End‘,
      ‘Alt Allele‘,
      ‘Ref Allele‘,
      ‘Associated Gene‘,
      OMIM,
      contains(″clinvar″))
  associated_gene <− pmda_annotation$‘Associated Gene‘ |>
    map_chr(~ str_split(.x, ″ //* ″) |>
    unlist( ) |>
parse_input <− function(data_path) {
  data <− read_lines(data_path)
  find_comments <− map_lgl(data, ~ str_detect(.x, ″#″))
  position <−
    which(find_comments == TRUE)[length(which(find_comments ==
    TRUE))]
  data <− read_tsv(data_path, skip = position) |>
    dplyr::filter(!str_detect(id, ″AFFX″) | str_detect(id, ″BGP″)) | >
    dplyr::rename(‘Probe Set ID‘ = id)
}
parse_annotation <− function(data_path) {
  pmda_annotation <− read_csv(data_path,
        skip = 20) |>
    dplyr::filter(‘dbSNP RS ID‘ != ″---″) |>
    dplyr::select(‘Probe Set ID‘,
      ‘dbSNP RS ID‘,
      Chromosome,
      ‘Physical Position‘,
      ‘Position End‘,
      ‘Alt Allele‘,
      ‘Ref Allele‘,
      ‘Associated Gene‘,
      OMIM,
      contains(″clinvar″))
  associated_gene <− pmda_annotation$‘Associated Gene‘ |>
    map_chr(~ str_split(.x, ″ //* ″) |>
```

In some embodiments, the system 200 may include a separate script for Clin Var due to the size of ClinVar. The script for Clin Var may be in python and may be executable on a high-performance computer or computing system.

The data parsers 208 may be configured to extract, transform, and load/maintain/store the datasets from each of the data sources 202 into a respective first slot of the database 204. In some embodiments, the data parsers 208 may be configured to map each of the datasets from the databases 204 to a respective first slot 210.

The data parsers 208 may be configured to generate a query for a subset of all data that is maintained at/by the respective data sources 202. For example, the data parsers 208 may be configured to generate a query for a subset of data points that are deemed clinically relevant. The data parsers 208 may be configured to generate a query for clinically relevant data points by requesting data corresponding to observations that have less than 75% missing information. The data parsers 208 may be configured to generate a query for clinically relevant data points by requesting data corresponding to observations which match at least one additional object slot. The data parsers 208 may be configured to generate a query for clinically relevant data points by requesting data corresponding to observations that are not duplicated across individual slots. In this regard, the data parsers 208 may be configured to generate a query for clinically relevant data points which are not filtered according to one or more of the following criteria: 1) observations that contain greater than 75% missing information, 2) observations that fail to match with at least one additional object slot, or 3) observations that are duplicated across individual slots.

The system 200 may include one or more data joiners 212. The data joiners 212 may be or include any device, component, element, or hardware designed or configured to join, reproduce, collect, or otherwise combine together annotated VCF files from the data parsers 208 into the database 204. In some embodiments, the joiners 212 files take the various annotated VCF files and joins them together into an efficient R S3 object. An S3 class may be the most prevalent and used class in R programming. An S3 object may be a list with its class attributes assigned some names. The member variable of the object created may be the components of the list. Each slot generated by or via the data joiners 212 may be associated with one of the databases, as depicted in FIG. 2 and FIG. 3. As such, the data joiners 212 may be configured to generate the S3 objects in an R programming language, which may be compatible with or consistent with an output from a diversity array 218, as described in greater detail below.

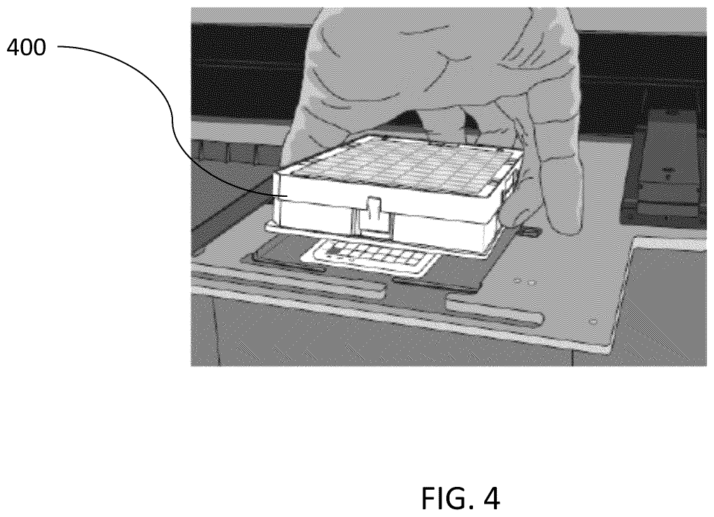

Referring back to FIG. 2 and FIG. 4, to perform a PGx test for a patient 100, the treating professional 102 may extract a sample 216 from the patient 100 which contains or includes DNA or genetic material of the patient 100. The treating professional 102 may extract the sample 216 in many different ways. For example, the sample 216 may include a blood sample, a saliva sample, a skin biopsy, a hair sample, a urine sample, a stool sample, a swab sample, to name a few possibilities. The blood sample may collected by inserting a needle into the patient's 100 arm and drawing a small amount of blood into a syringe or specialized collection device. The saliva sample is collected by asking the patient 100 to spit into a collection tube. The skin biopsy may performed by removing a small piece of skin tissue from the patient's body using a specialized cutting tool. The hair sample can be collected from the patient's 100 scalp or other areas of the patient's 100 body. The swab sample can collected by swabbing the inside of the patient's check or other areas of the body.

The system 200 may include a diversity array 218. The diversity array 218 may be or include any device, system, machine, or component designed or configured to detect and amplify a genetic material of interest from a sample 216. The diversity array 218 may include a microarray 400 and a number of probes. The probes may be immobilized on the microarray 400 surface. A technician may hybridize the sample 216 by mixing the sample 216 with the probes of the microarray 400. The probes may be configured to bind to specific genetic sequences of interest. When mixed, the genetic material of the patient's sample 216 may bind with the probes. The diversity array 218 may wash the microarray 400 to remove any unbound genetic material, thus leaving genetic material of the patient 100 that is bound to one of the respective probes. The diversity array 218 may scan the microarray 400 with a fluorescence detector to measure an expression of the genetic material (e.g., an expression of particular SNPs). The diversity array 218 may generate an output that indicates a presence or absence of specific gene or genetic sequences (SNPs) in the sample 216 as well as their respective expression levels (e.g., quantity and variations).

The system 200 may include a computing system 206. The computing system 206 may be configured to receive the output from the diversity array 218 (e.g., as an input 220). The computing system 206 may be configured to use the output to determine a treatment efficacy for various medication treatments based on the genetic makeup of the patient. In some embodiments, the computing system 206 may include various processors 222 and memory 224. For example, the memory 224 may store machine-readable instructions that, when executed by the processors 222, cause the processors 222 to perform various functions relating to determining the treatment efficacy for various medication treatments based on inputs received from the diversity array 218 and data from the database 204.

In some embodiments, the processor(s) 222 may be configured to identify the gene sequences of interest which are bound to specific probes of the microarray 400 (e.g., as identified based on the input 220 received from the diversity array 218). The input 220 may include unique identifiers of particular SNPs which were identified from the sample 216. For example, the unique identifiers may be rsIDs of the SNPs. The rsIDs may be reference single nucleotide polymorphism or reference SNPs, along with the corresponding number assigned in the dbSNP data source 202 (3).

The processor(s) 222 (e.g., drivers 226) may be configured to generate a query for the database 204 using the rsIDs from the input 220. The drivers 226 may be configured to extract, determine, or otherwise receive the relevant drug and phenotype information that are used to deliver the patient report 230. In addition, the drivers 226 may be configured to generate a query to slots 214 relating to the Pharm Var data source 202 (4), to convert all variants into star-allele haplotypes. Genetic variation may be known to influence the way in which individuals respond to therapeutics. Groups of variants that are inherited together, known as haplotypes, provide a basis for phenotype prediction and treatment decisions during pharmacogenomic testing. Accurately detecting functional haplotypes, popularly known as star (*) alleles, in clinically actionable pharmacogenes may thus be utilized for the implementation of personalized medicine. The driver(s) 222 may be configured to receive, retrieve, or otherwise obtain, from the database 204, a plurality of data entries which are related to the rsIDs in response to querying the database 204.

The computing system 206 may include one or more reporters 228 that are configured to generate, produce, or otherwise provide patient reports 230 based on the data from the database 204. In some embodiments, the reporters 228 may be configured to convert the star-allele driver file and create a patient report 230. The patient report 230 may be created in the R Markdown language, and can be outputted into HTML, or PDF. In this regard, because the second slots 214 have been encoded or otherwise reproduced in a format (e.g., R programming language) that matches the input 220 received from the diversity array 218, the processor(s) 222 may be configured to use the input 220 to directly query the database 204 without any further processing of the input 220, thereby conserving computing resources and reducing overall processing time. Additionally, because data from the multiple data sources 202 has been reproduced in the database 204, the processors 222 may query the database 204 rather than individually querying the data sources 202, thereby reducing a total number of API calls and reducing a likelihood of exceeding API call limits of the data sources 202. Further, because data from the plurality of data sources 202 has been reformatted into a common language, the processors 222 may query the database using a single query, thereby pulling all relevant data from the database 204 without generating multiple queries for the same rsID.

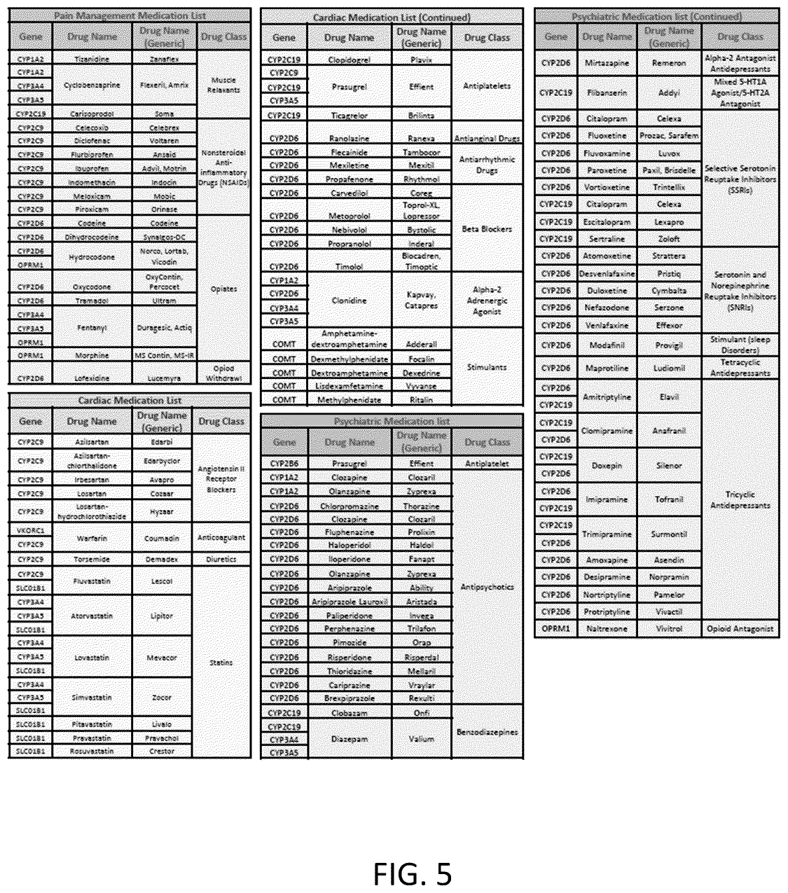

The processor(s) 222 (e.g., reporters 228) may be configured to receive treatment efficacy data from the database 204 in response to the query (or queries) that correspond to the input 220. The treatment efficacy data may be or include a treatment efficacy of medication treatments. The treatment efficacy may be or include a predicted responsiveness of particular medication treatment. The treatment efficacy data may be or include a table or list of medication treatments that can be used for prescribing to the patient, as illustrated in FIG. 5.

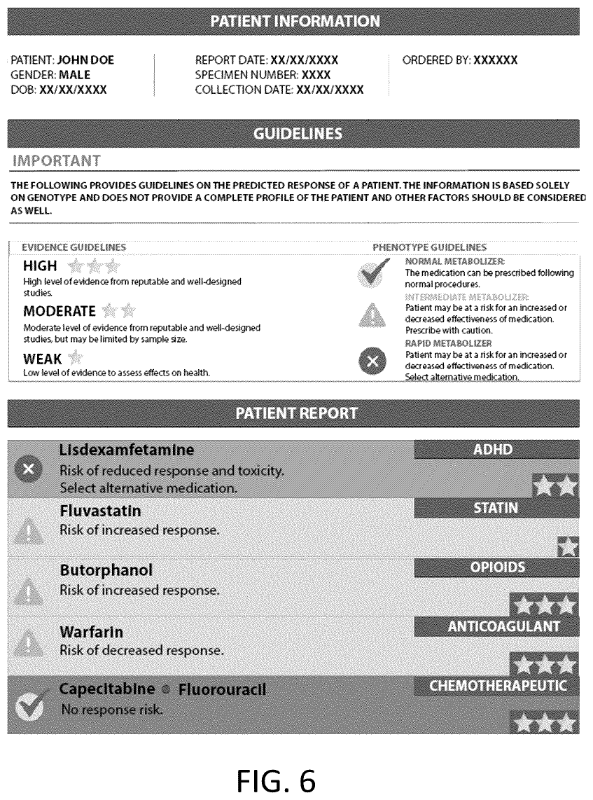

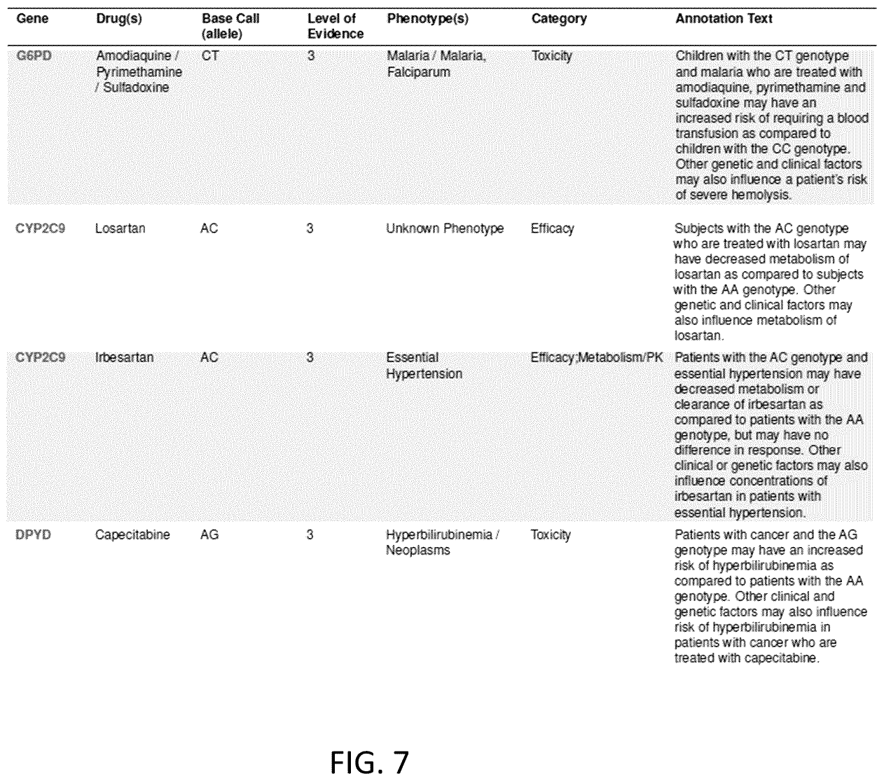

The reporters 228 may be configured to generate, produce, create, or otherwise provide a patient report 230 based on or according to the data obtained, retrieved, or otherwise received from the database. An example of the patient report 230 is illustrated in FIG. 6 and FIG. 7. In some embodiments, the reporters 228 may be configured to color-code the patient report 230 according to a risk profile of the patient to particular medication treatments (e.g., highlighted in red for high-risk medication treatment, highlighted in yellow for moderate-risk medication treatment, highlighted in green for low-risk medication treatment). Alternative color or highlight patterns may be utilized to distinguish different levels of risk and/or effectiveness for medication treatment. The reporters 228 may be configured to generate the patient report 230 using the compiled and curated data from the database 204 that is associated with each of the identified SNPs in the patient sample 216. As shown in FIG. 6, depending on certain risk profiles for medication (which itself is determined based on the SNPs identified in the patient sample 216), the reporters 228 may be configured to identify in the report 230 phenotype guidelines. The phenotype guidelines may be or include a metabolizer estimate or value that is determined or included in the database 204 associated with a particular SNP. Similarly, the reporters 228 may be congfigured to identify in the report 230 evidence guidelines based on which data source(s) 202 provided information related to the SNP identified in the patient sample 216. For some medication treatments, the patient report 230 may include a recommendation for alternative medication treatment as shown in FIG. 6. The patient report 230 may also include an appendix (as shown in FIG. 7), which may identify additional information relating to the treatment efficacy obtained from the database 204.

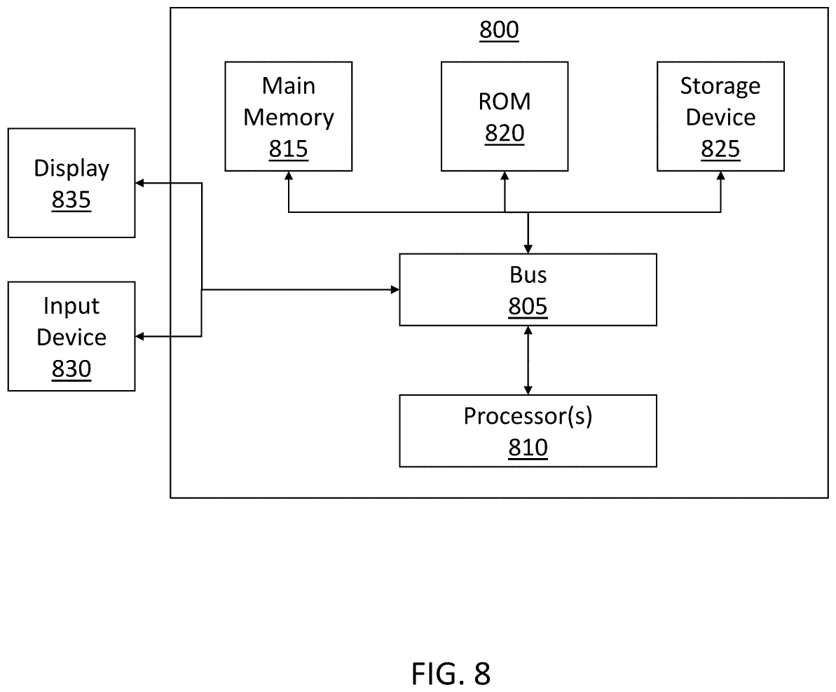

FIG. 8 depicts an example block diagram of an example computer system 800. Various components, elements, and/or hardware described herein (such as those described with reference to FIG. 1) may be implemented by, via, and/or on the computer system 800 including components thereof. The computer system or computing device 800 can include or be used to implement a data processing system or its components. The computing system 800 includes at least one bus 805 or other communication component for communicating information and at least one processor 810 or processing circuit coupled to the bus 805 for processing information. The computing system 800 can also include one or more processors 810 or processing circuits coupled to the bus for processing information. The computing system 800 also includes at least one main memory 815, such as a random access memory (RAM) or other dynamic storage device, coupled to the bus 805 for storing information, and instructions to be executed by the processor 810. The main memory 815 can be used for storing information during execution of instructions by the processor 810. The computing system 800 may further include at least one read only memory (ROM) 820 or other static storage device coupled to the bus 805 for storing static information and instructions for the processor 810. A storage device 825, such as a solid state device, magnetic disk or optical disk, can be coupled to the bus 805 to persistently store information and instructions.

The computing system 800 may be coupled via the bus 805 to a display 835, such as a liquid crystal display, or active matrix display, for displaying information to a user. An input device 830, such as a keyboard or voice interface may be coupled to the bus 805 for communicating information and commands to the processor 810. The input device 830 can include a touch screen display 835. The input device 830 can also include a cursor control, such as a mouse, a trackball, or cursor direction keys, for communicating direction information and command selections to the processor 810 and for controlling cursor movement on the display 835.

The processes, systems and methods described herein can be implemented by the computing system 800 in response to the processor 810 executing an arrangement of instructions contained in main memory 815. Such instructions can be read into main memory 815 from another computer-readable medium, such as the storage device 825.

Execution of the arrangement of instructions contained in main memory 815 causes the computing system 800 to perform the illustrative processes described herein. One or more processors in a multi-processing arrangement may also be employed to execute the instructions contained in main memory 815. Hard-wired circuitry can be used in place of or in combination with software instructions together with the systems and methods described herein. Systems and methods described herein are not limited to any specific combination of hardware circuitry and software.

Although an example computing system has been described in FIG. 8, the subject matter including the operations described in this specification can be implemented in other types of digital electronic circuitry, or in computer software, firmware, or hardware, including the structures disclosed in this specification and their structural equivalents, or in combinations of one or more of them.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device, etc.) may include one or more devices (e.g., RAM, ROM, Flash memory, hard disk storage, etc.) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or embodiment, and references to “an implementation,” “some implementations,” “one implementation” or the like are not necessarily mutually exclusive and are intended to indicate that a particular feature, structure, or characteristic described in connection with the implementation can be included in at least one implementation or embodiment. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Systems and methods described herein may be embodied in other specific forms without departing from the characteristics thereof. References to any terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining may be stationary (e.g., permanent or fixed) or moveable (e.g., removable or releasable). Such joining may be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References to “or” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

What is claimed is:

1. A computer-implemented method, comprising:
storing, by one or more processors in a non-transitory memory, a database comprising a plurality of slots, the slots respectively corresponding to data indicative of a gene marker and a treatment efficacy, the data indicative of the gene markers and treatment efficacies derived from a plurality of data sources;
receiving, by the one or more processors, an input extracted from a sample obtained from a patient, the input including a set of genetic sequences of the patient;
correlating, by the one or more processors, at least one of the set of genetic sequences from the input to a gene marker of a slot from the database, to determine a treatment efficacy of a treatment option for the patient; and
generating, by the one or more processors, a report indicating the treatment efficacy of the treatment option for the patient.

2. The method of claim 1, wherein the report identifies, for a plurality of conditions, a plurality of respective medication treatments, and for each respective medication treatment, a phenotype guideline indicating a predicted metabolizer and an evidence guideline indicating a degree of evidence from the database originating from a data source of the plurality of data sources.

3. The method of claim 2, further comprising:
determining, by the one or more processors, for a medication treatment of the plurality of respective medication treatments, that the phenotype guideline satisfies a threshold predicted metabolizer; and
generating, by the one or more processors, the report to prompt selection of an alternative medication treatment other than the medication treatment based on the phenotype guideline satisfying the threshold predicted metabolizer.

4. The method of claim 1, further comprising:
receiving, by the one or more processors, from a treating professional, an indication of a plurality of conditions of the patient and, for each of the plurality of conditions, a corresponding medication treatment identified by the treating professional for treating the respective condition.

5. The method of claim 4, wherein the report identifies, for the corresponding medication treatment for the plurality of conditions, a risk profile associated with the corresponding medication treatment.

6. The method of claim 5, wherein the risk profile for each medication treatment identifies a phenotype guideline indicating a predicted metabolizer and an evidence guideline indicating a degree of evidence from the database.

7. The method of claim 5, further comprising:
determining, by the one or more processors, the risk profile for each medication treatment by performing a look-up in the database using an identifier associated with a gene sequence of the set of gene sequences of the patient, to identify a gene marker matching the identifier associated with the gene sequence, the gene marker indicating an efficacy of the corresponding medication treatment for patients having the gene sequence.

8. The method of claim 1, further comprising populating, by the one or more processors, the database using the data compiled from the plurality of data sources.

9. The method of claim 8, wherein populating the database comprises:
extracting, by the one or more processors, datasets from the plurality of data sources, each dataset including a plurality of data points, each dataset from the plurality of data sources including at least some data of the plurality of slots;
compiling, by the one or more processors, the datasets from the plurality of data sources into a plurality of first slots, the data points of a respective dataset being assigned to a corresponding first slot of the plurality of first slots;
combining at least two slots into a grouped slot, based on a correspondence between a data point of one slot and another data point of another slot;
generating a plurality of second slots to include the plurality of first slots including the grouped slot; and
populating the database with the plurality of second slots.

10. The method of claim 9, wherein the input is extracted via a diversity array from the sample, the input having a language format, and wherein the plurality of second slots populated in the database has the language format that matches the input.

11. A system comprising:
a non-transitory memory storing a database comprising a plurality of slots, the slots respectively corresponding to data indicative of a gene marker and a treatment efficacy, the data indicative of the gene markers and treatment efficacies derived from a plurality of data sources;
one or more processors configured by machine-readable instructions to:
receive an input extracted from a sample obtained from a patient, the input including a set of genetic sequences of the patient;
correlate at least one of the set of genetic sequences from the input to a gene marker of a slot from the database to determine a treatment efficacy of a treatment option for the patient; and
generate a report indicating the treatment efficacy of the treatment option for the patient.

12. The system of claim 11, wherein the report identifies, for a plurality of conditions, a plurality of respective medication treatments, and for each respective medication treatment, a phenotype guideline indicating a predicted metabolizer and an evidence guideline indicating a degree of evidence from the database originating from a data source of the plurality of data sources.

13. The system of claim 12, wherein the one or more processors are further configured by machine-readable instructions to:
determine, for a medication treatment of the plurality of respective medication treatments, that the phenotype guideline satisfies a threshold predicted metabolizer; and
generate the report to prompt selection of an alternative medication treatment other than the medication treatment based on the phenotype guideline satisfying the threshold predicted metabolizer.

14. The system of claim 11, wherein the one or more processors are further configured by machine-readable instructions to receive, from a treating professional, an indication of a plurality of conditions of the patient, and, for each of the plurality of conditions, a corresponding medication treatment identified by the treating professional for treating the respective condition.

15. The system of claim 14, wherein the report identifies, for the corresponding medication treatment for the plurality of conditions, a risk profile associated with the corresponding medication treatment.

16. The system of claim 15, wherein the risk profile for each medication treatment identifies a phenotype guideline indicating a predicted metabolizer and an evidence guideline indicating a degree of evidence from the database.

17. The system of claim 15, wherein the one or more processors are further configured by machine-readable instructions to:
determine the risk profile for each medication treatment by performing a look-up in the database using an identifier associated with a gene sequence of the set of gene sequences of the patient, to identify a gene marker matching the identifier associated with the gene sequence, the gene marker indicating an efficacy of the corresponding medication treatment for patients having the gene sequence.

18. The system of claim 11, wherein the one or more processors are further configured by machine-readable instructions to populate the database using the data compiled from the plurality of data sources.

19. The system of claim 18, wherein, to populate the database, the one or more processors are configured by machine-readable instructions to:
extract datasets from the plurality of data sources, each dataset including a plurality of data points, each dataset from the plurality of data sources including at least some data of the plurality of slots;
compile the datasets from the plurality of data sources into a plurality of first slots, the data points of a respective dataset being assigned to a corresponding first slot of the plurality of first slots;
combine at least some slots into a grouped slot, based on a correspondence between a data point of one slot and another data point of another slot;
generate a plurality of second slots to include the plurality of first slots including the combined group slot; and
populate the database with the plurality of second slots.

20. A non-transitory computer readable medium storing instructions that, when executed by one or more processors, cause the one or more processors to:
access a database comprising a plurality of slots, the slots respectively corresponding to data indicative of a gene marker and a treatment efficacy, the data indicative of the gene markers and treatment efficacies derived from a plurality of data sources;
receive an input extracted from a sample obtained from a patient, the input including a set of genetic sequences of the patient;
correlate at least one of the set of genetic sequences from the input to a gene marker of a slot from the database to determine a treatment efficacy of a treatment option for the patient; and
generate a report indicating the treatment efficacy of the treatment option for the patient.
